(19) United States
(12) Patent Application Publication
(10) Pub. No.: US 2025/0339234 A1
(43) Pub. Date: Nov. 6, 2025

(54) PROCESSOR CONTROLLED SURGICAL STEP STOOL WITH AUTOMATIC HEIGHT ADJUSTMENT AND ELECTRONIC FOOT SWITCHES

(71) Applicant: Smart Step Surgical LLC, Billings, MT (US)

(72) Inventors: Jennifer Lauren Quon, Toronto (CA); Frank William Crawford, Billings, MT (US); Casey M. Chambers, Boise, ID (US)

(21) Appl. No.: 19/268,847

(22) Filed: Jul. 14, 2025

Related U.S. Application Data
(63) Continuation-in-part of application No. 17/711,226, filed on Apr. 1, 2022, now Pat. No. 12,357,419.

(51) Int. Cl. A61B 90/60; A47C 12/00

(52) U.S. Cl. CPC A61B 90/60; A47C 12/00

(57) ABSTRACT

Operating room tables have limited height adjustment capability. To adequately treat a patient, surgeons often have to risk back injury and/or stand on rickety piles of prior art step stools, which is unsafe and can compromise sterility. Here, a computer-processor-controlled, motorized, surgical step-platform device is disclosed. The device is configured to have dimensions similar to a step stool. The device is configured to easily slide across a floor to an operating table. Then, either by a built-in user interface, or by remote control from a Smartphone, the device can affix itself into position and automatically raise between 5 to 18 inches above the floor. The device has sophisticated rechargeable battery power management and further accommodates wired, and wireless foot switches to control nearby surgical equipment.

### CROSS REFERENCE TO RELATED APPLICATIONS

This application is a continuation in part of U.S. patent application Ser. No. 17/711,226, filed Apr. 1, 2022, the entire contents of which are incorporated herein by reference.

### BACKGROUND OF THE INVENTION

### Field of the Invention

This invention is in the field of adjustable stools and automated surgical equipment.

### Description of the Related Art

To prevent back injury and degeneration, surgeons must position themselves ergonomically during long surgeries. However, surgeons, their assistants, and surgical technicians also need to optimize their positions around the surgical table to visualize essential patient anatomy, leverage surgical tools and equipment, and compensate for differences in the surgeon's heights. Thus, surgeons face a constant conflict between avoiding personal injury and optimizing patient treatment.

Unfortunately, surgical tables (such as operating tables) are limited in their height adjustment parameters. They often cannot be adjusted to the heights needed for surgeons to perform a given surgery effectively. To optimize visualization and equipment use, surgeons will often stand on step stools throughout the entire operative case. In a typical surgery, at least one member of the operating team will require 1-3 steps due to height discrepancies amongst team members. Surgical step stools, produced by Pedigo Products, Vancouver Washington, and other manufacturers, are thus a widespread piece of equipment in the operating room. Surgeons from all subspecialties use them.

Current step stools vary in material (plastic, metal, etc.), structure, and size/weight but are non-adjustable. They are similar to the types used in warehouses and kitchens to reach high shelves.

Therefore, steps are stacked if additional height is required. For example, a Pedigo stackable footstool will typically have a height of about six inches, a depth of about 14 inches, and a width of about 19 inches. It is common to stack between one to three footstools on top of each other to achieve heights between 6 inches and 18 inches.

However, if the steps are not stacked properly, they can be unsteady while surgeons stand on them. Some prior art step stools include handles that allow the steps to interlock side-by-side to increase safety and sturdiness for the surgeon standing on the step. These features also make the surgical step stools easier for staff to pick up and maneuver.

Surgeons typically use several (2-3) ancillary electrical pedal switches connected to operating room equipment and surgical tools during surgery. These tools include high-speed drills, electrocautery apparatus, harmonic bone scalpels, and other equipment types. These foot pedals need to be within range of the surgeon's feet. If the surgeon is standing on a step, they will need additional platform space to accommodate these pedal switches. As a result, multiple prior art step stools may be placed or stacked side-by-side to get this space.

Often, a surgeon may need to combine 4-6 different step stools to accommodate the height and platform space required to control multiple pedal switches. The other staff members must move and stack these steps each time a surgical team member moves around the operative table. This can create a workplace hazard.

Another problem is that pedal switches are often accidentally kicked off these step stools. A non-sterile team member (circulating nurse or other operating room staff) must place the pedal back onto the platform when this happens. This practice increases the chance of contaminating the surgical field as non-sterile team members encroach upon the sterile field (within 1-2 feet) to adjust these pedals. The cords connecting these pedals can also become tangled as the pedals are moved and angle adjusted.

Examples of prior art step stools with handles include Sandel and Ramon D632,101. Brown taught a motorized adjustable step stool in U.S. Pat. No. 5,285,992.

### BRIEF SUMMARY OF THE INVENTION

The invention is based, in part, on the insight that what is needed is an improved automatically adjustable standing platform to optimize surgical performance and operating convenience. Ideally, such a platform should be automatically or semi-automatically height-adjustable (such as between about 5″ high to 15″ high, or 6″ high to 18″ high) and accommodate (either directly, such as by having somewhat wider dimensions such as 17″ deep and 27″ wide, and/or via an optional “kick-out” platform) various pedal foot-switches to control operating room equipment. Such a platform would support surgeons and surgical assistants while performing surgery.

In this disclosure, the terms “step-platform” and “stepstool” will occasionally be used interchangeably.

As will be discussed, in some embodiments, the invention may be a computer-processor-controlled, motorized, step-platform device. The invention is configured to have dimensions similar to a step stool, and to elevate the operator (often surgeon or surgical assistant) to various distances, such as between about 5 inches and 18 inches, above the operating room floor, thus enabling the operator (surgeon), often while operating on a patient on a surgical table such as an operating table, to easily adjust for operator-to-operator variations in height or other operator body dimensions (e.g., arm length, lower limb dimensions, and the like).

This step-platform device can comprise a lightweight (under 40 pounds, and usually under 40 pounds) motorized step-platform device. For easy cleaning, operating room floors are usually smooth and usually lack bumps or crevices. The underside of the invention's step platform may have at least some areas configured with smooth plastic or metal that serve as low friction skids even without any optional wheels. Due to its light weight, the invention's step-platform device can be moved or skid along smooth (flat and uncarpeted) floors by one person (either pushing or carrying), and will often be initially placed in a first desired position proximate a table such as an operating table. Here assume that the invention's step platform will be moved while in a contracted form (e.g., only about 5 to 7 inches high).

As will be discussed in more detail shortly, the invention's motorized step-platform device will typically comprise at least one processor (often an ARM-based, x86 based, 8051 based, or other type microprocessor or microcontroller). The step-platform device will also typically comprise memory (often RAM, or FLASH memory), any of a wired or wireless data interface, a motorized height adjustment device (usually at least one processor controlled motor or other type electronic actuator (306), along with ancillary mechanical devices to translate motor/actuator movement into a step-stool height adjustment), a top-plate (preferably a customizable top plate, that can be configured to hold one or more electronic pedal switches), and optionally, often at least one motorized bottom suction device. Although optional, this suction device can be activated during use, and can significantly reduce the chance that the step-platform device will accidentally move during use.

The invention's step-platform device can be configured to raise and lower, typically between about 5″ and about 18″ high, using at least one processor-controlled motor and either external commands (e.g., commands sent by direct input onto a data interface such as various switches or touch-screens mounted on the step-platform device itself, a wired computer interface such as a USB interface, or a wireless transceiver configured to receive wireless commands sent from local smartphones or other wireless devices), or in response internal commands, such as height adjustment data stored in memory).

To do this, the step-platform device's at least one processor is typically configured to use input from either its internal memory or from its previously discussed data interface to optionally immobilize and then height-adjust the step-platform device to a first preferred height (e.g., the height preferred by a given operator intending to use the step-stool, which may be different from the step-platform device's previous height). This is done by commanding the motorized height adjustment device to adjust the height of the step-platform device. The optional (but preferred) “immobilization” step may be done by commanding the step-platform's optional motorized bottom suction device to generate a partial vacuum against the (smooth) floor. Alternatively, alternative methods, such as retractable ball casters may be used.

In a preferred embodiment, “immobilization” means that the step-stool device will adhere to the floor tightly enough that casual application of pressure, such as 20-100 pounds or less of sideways pressure, will not cause the step-platform device to accidentally move.

After use, it will often be desirable to reconfigure the step-platform to take up less room, and also to move it to a different location for storage or later use. To do this, the step-platform device's at least one processor can be further configured to use input from the data interface to command the optional motorized bottom suction device to release any partial vacuum. This will cause the device to release its grip on the smooth floor. Alternatively, the operator may step off of the device, and springs on the retractable ball casters can force the rotating balls out of their holders, thus enabling the step stool to regain mobility.

This in turn enables a much smaller amount of external force, often 20 pounds or less, which can be readily applied by one person, to move the motorized step-platform over the floor to a different location. Additionally, the at least one step-platform processor can be further configured to use input from the data interface, or other source (such as the device memory) to command the motorized height adjustment device to adjust the height of the step-platform to a different height, such as a lower (5 to 7 inch high) storage height.

Other embodiments, including interfacing with various types of electrical foot pedal switches, built-in batteries, power management, and the like will also be discussed.

### BRIEF DESCRIPTION OF THE DRAWINGS

FIG. 1 shows a top view of the surgical step stool (step-platform) in a retracted configuration.

FIG. 2 shows a bottom view of the surgical step stool, showing the bottom suction devices.

FIG. 3 shows a top view of the surgical step stool in an extended configuration.

FIG. 4 shows a detail of a human user, such as a surgeon, stepping on top of the surgical step stool.

FIG. 5 shows an example of the surgical step stool in use.

FIG. 6 shows a side view of some of the interior components of the step stool.

FIG. 7 shows an overview of how the device's processor controls the surgical step stool.

FIG. 8 shows how the customizable top plate may be removed and replaced by an alternative customizable top plate.

FIG. 9 shows a top view of the step stool with some of the side housing removed.

FIG. 10 shows a top view of the step stool with both the side housing and top plate removed.

FIG. 11 shows a top view of the step stool with the side housing, top plate, battery pack, and electronics housing removed.

FIG. 12 shows a detail of the position of the vacuum pump components of the step stool's two motorized suction devices.

FIG. 13 shows a top view of an alternate embodiment of the surgical step stool, here with a simplified top plate, and a foot-activated user interface comprising at least two switches. Note the slots in the upper telescoping skirt, which can accommodate tabs to mount optional side platform wings as shown in FIG. 14.

FIG. 14 shows a top view of an alternate embodiment of the surgical step stool previously shown in FIG. 13, here further comprising two optional side platform wings. These side platform wings can be used to hold foot pedals, which may be used to control other operating room equipment.

FIG. 14A shows a detail of the right-side platform wing, previously shown in FIG. 14.

FIG. 15 shows a bottom view of the alternate embodiment of the surgical step stool. In this alternate embodiment, the motorized bottom suction device is optional. Instead, the surgical step stool is supported by at least three retractable ball casters.

FIG. 16 shows a detail of one of the retractable ball casters previously shown in FIG. 15. Here each retractable ball caster mounted in a holder, and configured with a spring arrangement that allows the retractable ball caster to extend from the holder and the bottom plate when no operator is on the step stool (e.g. operator weight less than a preset amount). This allows the step stool to be moved or slid to a position as desired. The spring arrangement is further configured so that when an operator stands on the step stool (e.g., with an operator weight exceeding a preset amount), the ball caster retracts into the holder. This increases friction between the step stool and the floor, impeding further motion of the step stool.

FIG. 17 shows an interior detail of the retractable ball caster, showing the spring arrangement.

FIG. 18 shows examples of the step stool in the retracted and extended configurations. Note that in this example, the step stool has three telescoping skirts.

FIG. 19 shows an exploded diagram of the alternate embodiment of the invention previously shown in FIG. 13.

FIG. 20 illustrates one example of how a height sensor, comprising an optical time-of-flight sensor in this case, may operate. In this example, the time-of-flight sensor is mounted proximate to the device's bottom plate, shines a light on the bottom side of the top plate, and determines height by measuring the time it takes for the reflected light to return to the time-of-flight sensor. Other methods of determining height, such as measuring a change in position of the motorized actuator, may also be used.

### DETAILED DESCRIPTION OF THE INVENTION

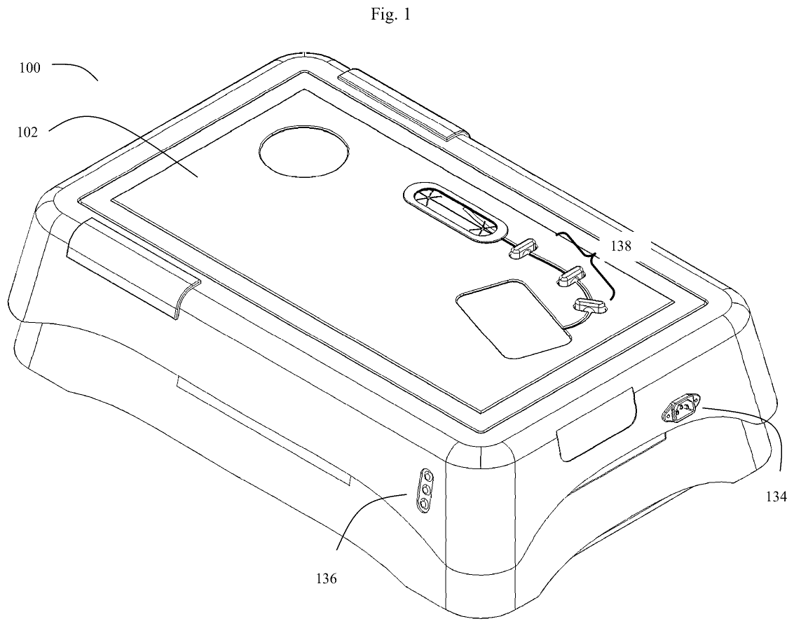

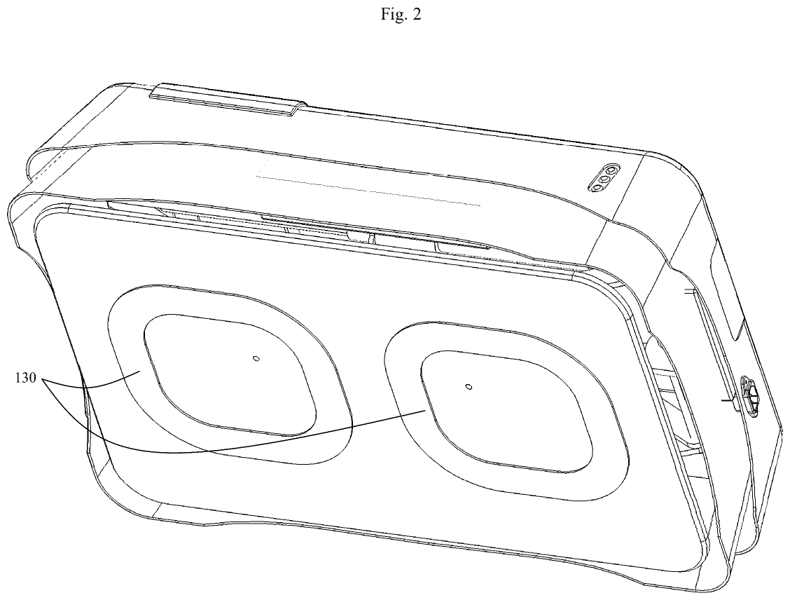

FIG. 1 (100) shows a top view of the surgical step stool (step-platform) in a retracted configuration (100). A top plate (102), AC power input (134, which can be used to either power the step-stool, or recharge the step-stool batteries), and status lights (136, such as LED charge indicator lights) are also shown. See also FIG. 2 to FIG. 5.

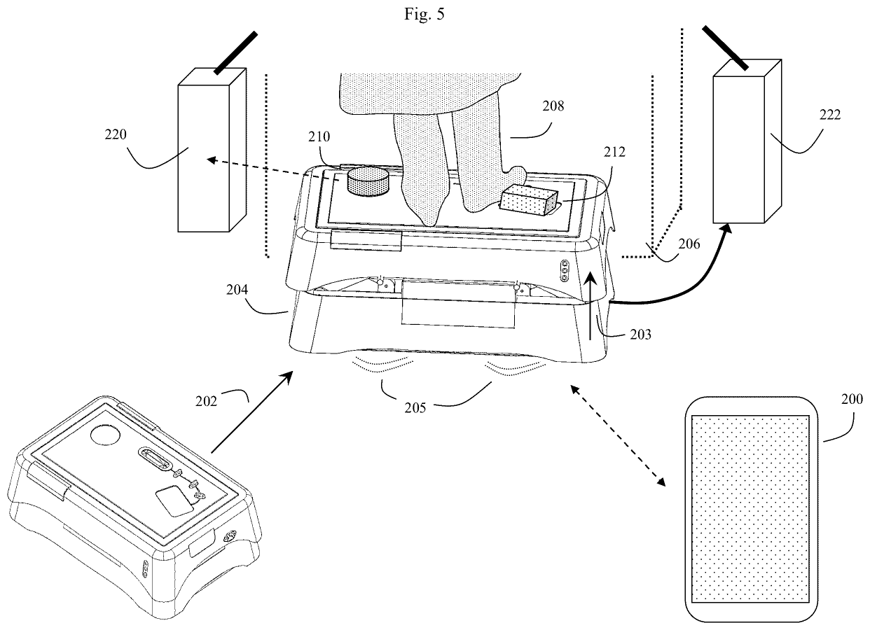

An optional cable management system (138), which may comprise various spring-loaded cable claims intended to work with a wired foot switch (see FIG. 5, 212), is also shown. This cable management system (138) functions to provide a plurality of integrated cord traps that are useful for corded foot pedal switches. This helps reduce tangling and clutter from the cords, as well as to help secure the corded foot pedals and prevent them from getting kicked off the platform. An added benefit as this helps improve sterility, as it helps to minimize the number of times that non-sterile personnel need to approach the sterile field.

In some embodiments, the invention may be a motorized step-platform device (100) configured to adjust to operator-to-operator variations in operator height and other operator parameters. This step-platform device is often referred here as a surgical step stool, or “step stool”. This device is lightweight (typically under 50 pounds weight, and preferably under 40 pounds weight for easy mobility). In at least one mode, as shown in FIG. 5, the motorized step-platform device is configured to slide (202) along a smooth floor, such as an uncarpeted operating room floor, by an application of external force (usually human hand or foot force) to a first desired position (204) proximate a table, such as an operating table. The base of this operating table is shown as (206).

Here “proximate” means that a human (208) standing on the step-platform is within arms-length of at least a portion of the table, in other words less than about two feet from the table (206).

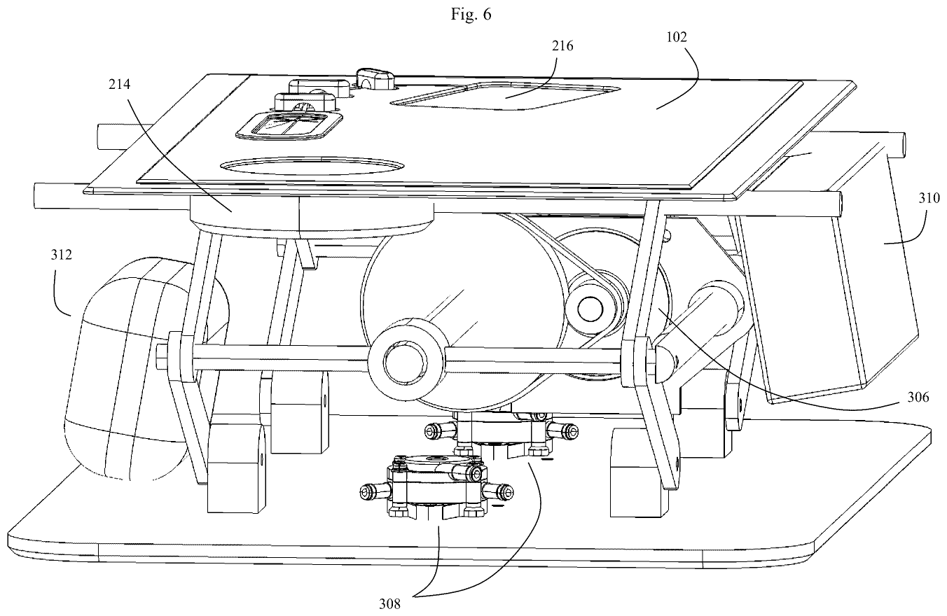

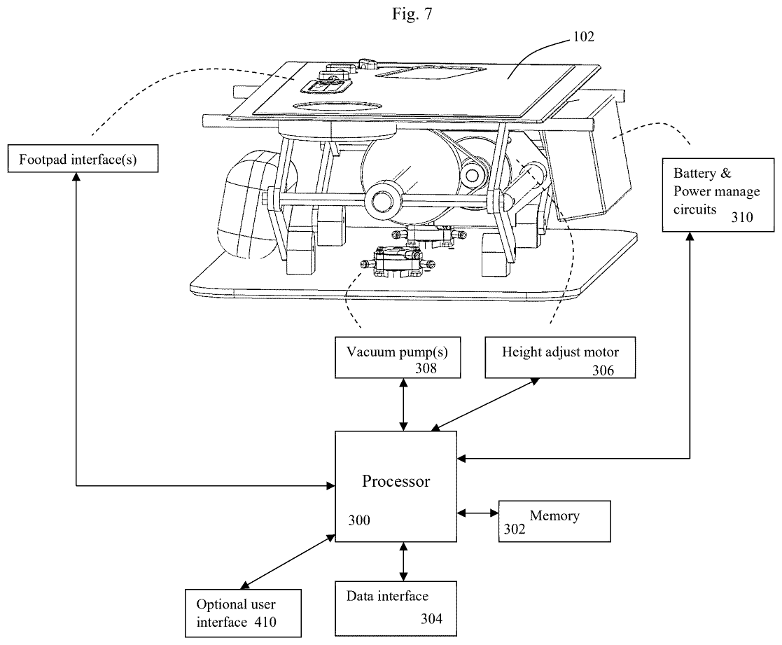

As shown in FIG. 1-5, as well as FIG. 6-7, this motorized step-platform device typically comprises at least one processor (300), memory (302), data interface (here exemplified by a wireless transceiver, such as Bluetooth transceiver 304), motorized height adjustment device (which will usually have at least one height adjust motor 306 or actuator, along with other mechanical equipment such as belts, gears, lift arms, link rods, and the like), customizable top plate (102), and one or more optional motorized bottom suction devices (see FIG. 2, 130), usually driven by at least one vacuum pump (308).

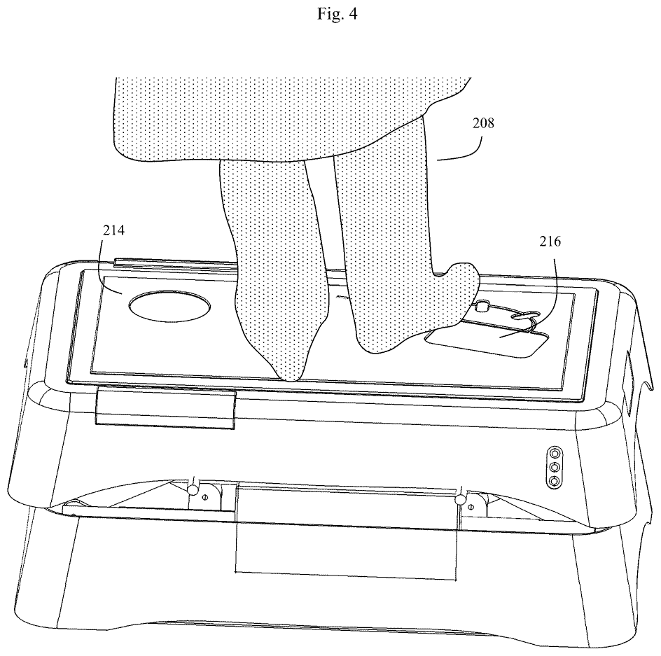

In some embodiments, the least one processor (300) may be configured (300) to use input from either the computer memory (302) or from the data interface (304) to immobilize and/or height-adjust the step-platform device to a first height. The processor does the height adjust by commanding the motorized height adjustment device (usually by height adjust motor 306) to adjust the height of at least the top plate (102) of the step-platform device. In some embodiments, the processor may further implement an immobilization step by commanding one or more optional motorized bottom suction devices (usually by vacuum pumps 308) to generate a partial vacuum against the floor. These optional suction devices fix the step-stool to the floor so that it will usually take more than 100 pounds of force to move the step stool, thus effectively immobilizing it, and allowing a human operator, such as a surgeon (208), to safely mount the stepstool, as shown in FIG. 4 and FIG. 5, without worry that the step stool might accidentally shift position when someone steps on top of it.

In some embodiments, when the user(s) wish to move the step-stool to a different location, if the optional suction devices are being used, the at least one processor (300) may be further configured to use input from the data interface (304) or an alternative vacuum release switch to command the optional motorized bottom suction devices (often via vacuum pump 308 or computer-controlled release valves) to release the partial vacuum. Once the vacuum is released, the step-stool is no longer tightly bound to the floor. Since it is inherently light weight (usually under 40 pounds), this enables external force (such as human arm or foot force) to move the motorized step-platform over the floor to a different location, such as a storage location, or different location around the table (206). The at least one processor (300) may be further configured to use input from the data interface (304) to command the motorized height adjustment device (usually by height adjust motor 206) to adjust the height of the step-platform to a different height.

FIG. 2 shows a bottom view of the surgical step stool, showing the bottom suction devices (130).

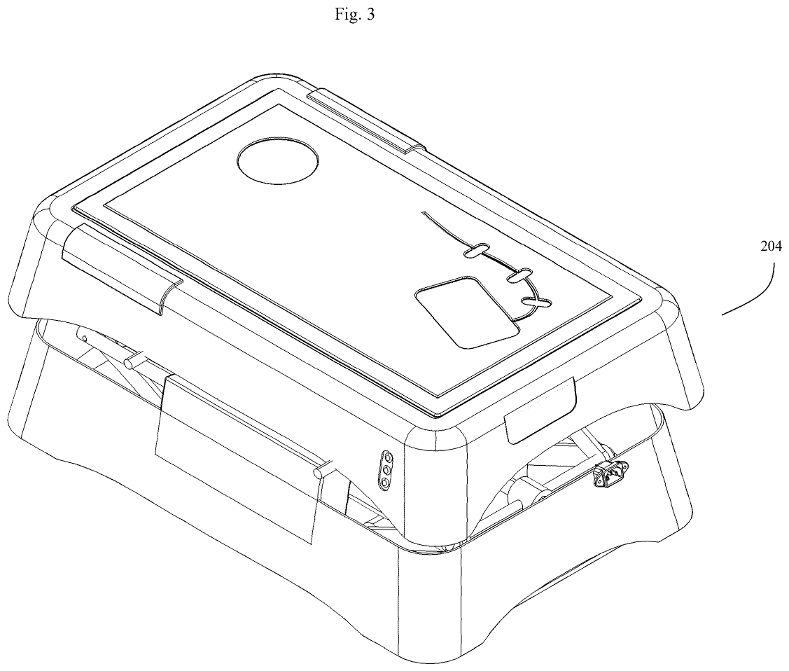

FIG. 3 shows a top view of the surgical step stool in an extended configuration (204) that has a height higher than the height previously shown in FIG. 1.

FIG. 4 shows a detail of a human user, such as a surgeon (208), stepping on top of the surgical step stool. The top of this step stool has two defined locations (214, 216) that can accommodate a wireless foot switch (214) or a wired foot switch (216). In some embodiments, at least one wireless foot switch location, such as (214), may further comprise an indictive charging station, such as a Qi or other wireless power transfer unit, to recharge certain types of wireless foot switches.

FIG. 5 shows an example of the surgical step stool in use. Here assume that the step stool was originally stored away from an operating table in an inactive and contracted configuration. The step stool is first moved (202) to a desired location next to an operating table (206). The step stool is then powered on using commands from a data interface (304), or optional built-in user interface, often by either receiving user commands over the built-in user interface, or by receiving Bluetooth wireless commands from a suitably configured smartphone (200). The step stool (204) may optionally be commanded to affix itself firmly to the floor using the optional vacuum pumps (FIG. 7, 308) in its optional bottom mounted motorized suction device (130) to generate a partial vacuum (exemplified by curved dotted lines 205). The step stool is also commanded to raise (adjust its height upward 203). In this example, two different electronic foot switches (210, 212) have been mounted at two defined locations on the footstool's top plate (see FIG. 4, 214, 216). By stepping on these foot switches, the surgeon can then control various types of equipment. This equipment can include operating room equipment, exemplified by (220) and (222).

FIG. 6 shows a side view of some of the interior components of the step stool, including the motor portion of the device's motorized height adjustment device (306), motorized vacuum pumps (308) (part of the device's motorized bottom suction device 130), customizable top plate (102), battery pack (310), and other components, such as an electronics housing (312) that may house the processor (300) and various other electronic components (e.g., 302, 304, etc.)

FIG. 7 shows an overview of how the device's processor (300) controls the surgical step stool's optional motorized bottom suction device (via optional vacuum pump 308), motorized height adjustment device (via height adjust motor 306), batteries and power management circuits (310), and other components. The processor may store various settings in memory (302), and can also be controlled using a user interface or data interface (304). In some embodiments, this can be by wireless connection to an external computerized device such as a smartphone (FIG. 5, 200). The data interface can also include optical or wireless data interfaces, such as USB connectors, electrical switches, built-in-touchpads, touch sensitive screens, keypads, and the like. The data interface can also include various foot or hand activated switches as well.

Expressed in methods format, the invention can also be viewed as a method of configuring equipment (such as an operating room table 206) to adjust to operator-to-operator (208) variations in height and other operator physical parameters.

This method can comprise using external force to slide (202) a motorized step-platform (100) along a floor to a first desired position proximate a table (206). As previously discussed, this motorized step-platform can comprise at least one processor (300), memory (302), data interface (304), motorized height adjustment device (306), customizable top plate (102), and optional motorized bottom suction device(s) (130, 308).

The method uses the at least one processor (300) and input from either the memory (302) or from the data interface (304) to immobilize (205) and height-adjust (203) the step-platform to a first height by commanding the motorized height adjustment device (306) to adjust the height of the step-platform, and optionally commanding the at least one motorized bottom suction device (308) to generate a partial vacuum against the floor.

According to the method, when desired, input from the data interface (304) or user interface, and the at least one processor (300) can optionally be used to command the optional motorized bottom suction device to release any partial vacuum (using by using vacuum pump 308 or a release valve). This breaks the suction (if any) that was previously preventing sideways movement, thus enabling the external force to move the motorized step-platform over the floor to a different location (reverse of 202). Further, as desired (and often to either assist in storage or reconfigure for a different user), input from the data interface (304) or user interface, and the at least one processor (300) can be used to command the motorized height adjustment device (306) to adjust the height of the step-platform to a different height (such as 203).

Although in this disclosure, we will generally refer to the invention as a device, the methods version of the invention is not disclaimed, and it should be readily apparent how the device version and the methods version are essentially one and the same invention.

The optional user interface will be described in more detail shortly.

In some embodiments, the data interface (304) can be any of a wired data interface, such as a universal serial bus (USB), touchscreen, or various step-stool located control switches. Alternatively, or additionally, the data interface (304) can be a wireless data interface, such as a Wi-Fi and/or or Bluetooth wireless transceiver. In a preferred embodiment, the data interface (304) includes at least one wireless transceiver, and the at least one processor (300) and the data interface (304) are configured to receive commands and data from an external computerized device. Alternatively, as will be discussed shortly, an optional user interface (410) may be used to interact with the processor.

For example, in some embodiments, as shown in FIG. 5, the data interface (304) can comprise a wireless Bluetooth transceiver, and the external computerized device can comprise a handheld computerized device (200), such as a smartphone or tablet computer, that itself further comprises a touch-screen and a Bluetooth transceiver.

The processor (300) and data interface (304) can be configured (usually by software stored in memory 302) to operate in various ways. For example, in some embodiments, the at least one processor (300) and the data interface (304) can be configured to transmit or receive operator (that is a user, such as a surgeon or surgical technician) specific parameters from memory (302). These operator specific parameters can comprise operator preferred step-platform height parameters. So, a medium height surgeon may can store “medium extension” step-platform height parameters in memory, while a short surgeon may store “maximum extension” step-platform height parameters in memory. In situations where the data interface (304) is a Bluetooth transceiver, and the processor (300) is configured to receive Bluetooth commands from a suitably configured smartphone (200), then a surgeon or attendant may merely call up a smartphone step-stool app, enter in any security codes as needed, and command the step-stool to extend to “Dr. Smith's previously stored parameters of 13 inches in height.”

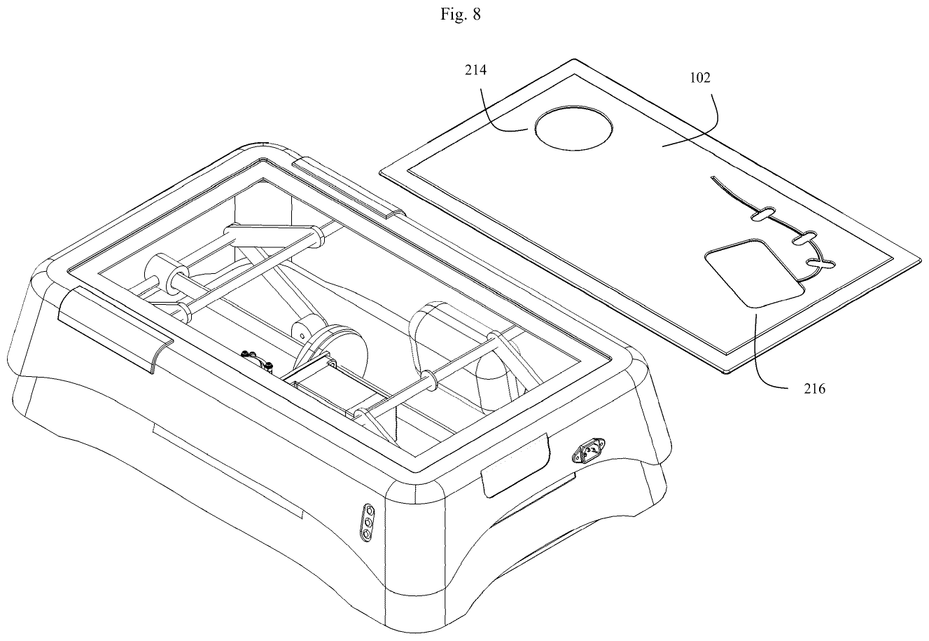

FIG. 8 shows how the top plate may be a customizable top plate (102), which may be removed and replaced by an alternative customizable top plate. This embodiment, this customizable top plate (102) and the step-platform may be configured to enable toolless (e.g., one-touch, quick release) attachment and detachment of the customizable top plate from the step-platform.

In some embodiments, this customizable top plate may further comprise at least one defined location (such as 214 and 216). These locations are typically configured to interface with an electronic foot switch (such as FIG. 5, 210, 212), that in turn is configured to control at least one item of electronic equipment (FIG. 5, 220, 222).

In other embodiments, some of the electronic foot switches may be mounted on side platform wings. In some embodiments, such side platform wings may be attached or detached from the step platform device as desired.

In some embodiments, the foot switch interfaces (214, 216) can comprise any of a countersunk depth configured to accommodate the electronic foot switch (210), a wireless charger configured to recharge (or supply power to) an electronic foot switch (210), and/or a wired jack or plug configured to receive wired electrical signals from the electronic foot switch (212). Thus, in a common case where the table (206) is an operating table, the electronic foot switches (210, 212) may be configured to control operating room equipment (220, 222).

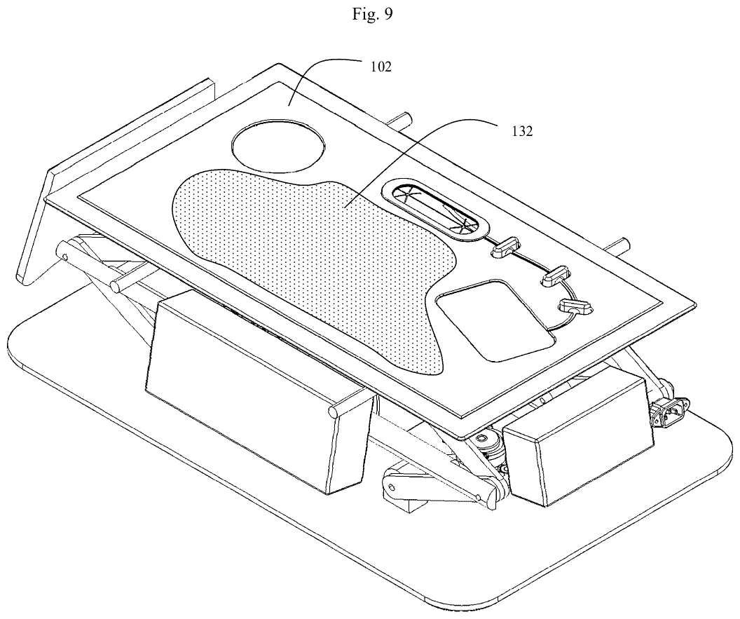

FIG. 9 shows a top view of one embodiment of the step stool with some of the side housing removed. This figure also shows that in some embodiments, at least a portion of the customizable top plate (102) further comprises an anti-slip mat (132). Such anti-slip mats can comprise a textured surface, often comprising synthetic rubber or other slip resistant material.

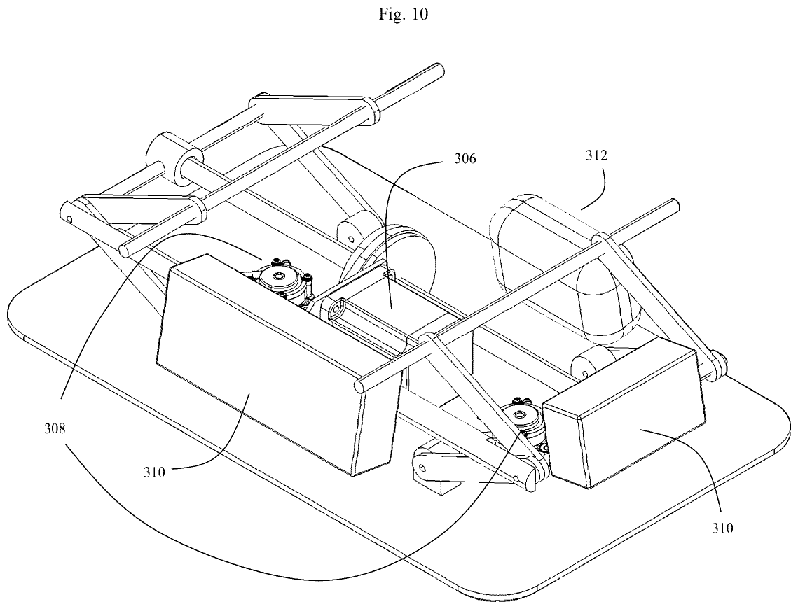

FIG. 10 shows a top view of the step stool with both the side housing and top plate removed. This exposes more details of the step stool's motorized height adjustment device (e.g., motor 306), optional motorized bottom suction device (e.g., vacuum pumps 308), battery pack (310) and an optional second battery pack or AC power converter (311), and electronics housing (312). This view also exposes other components of the height adjustment device.

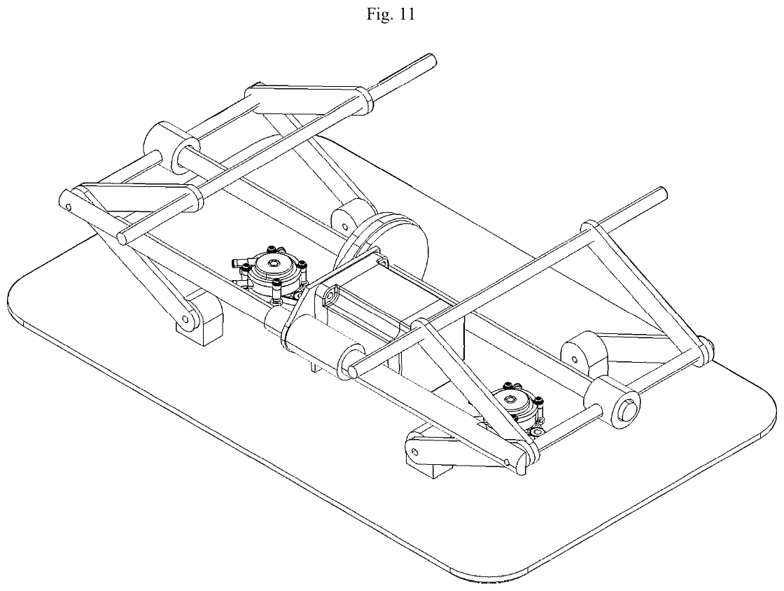

FIG. 11 shows a top view of the step stool with the side housing, top plate, battery pack, and electronics housing removed, exposing more details of the motor and lift link rod components of the motorized height adjustment device, as well as the optional vacuum pump components of the optional motorized bottom suction devices. Examples of such lift rod and motor arrangements may be found in Brown, U.S. Pat. No. 5,285,992, the entire contents of which are incorporated herein by reference.

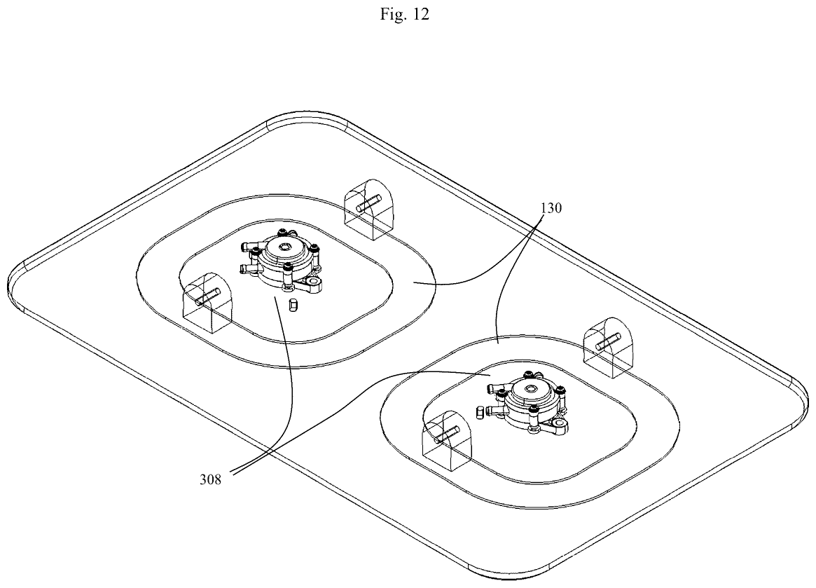

FIG. 12 shows a detail of the position of the optional vacuum pump components (308) of the optional motorized suction devices (130). In this view, the bottom of the step stool is shown as transparent, enabling the flexible membrane components of the optional motorized bottom suction devices (130) to be better visualized. Some base supports of the motorized height adjustment device are also shown.

In some embodiments, the motorized step-platform (100) can comprise at least one rechargeable battery (310), (such as a lithium-ion battery or other type rechargeable battery) and battery access port. In a preferred embodiment, this at least one rechargeable battery and battery access port may be configured to enable toolless exchange of the at least one rechargeable battery.

In some embodiments, the motorized step-platform may also be configured with a second rechargeable battery, with power supply electronics configured to enable a toolless hot-swap of one rechargeable battery while the motorized step-platform is operating with the other rechargeable battery. This would enable battery changes without impact on any of the step-platform height, the motorized bottom suction device, or electronic foot switches. Alternatively, the step-platform may be plugged int an AC electrical outlet via an AC power input plug (134)

In some embodiments, to extend the area of the top plate further, the step-platform may further include one or more hinged fold-out (or kick-out) or detachable drop-leaves on either side of the set-platform that can be extended to increase the area of the top-plate when needed, and then can be folded back against the sides of the set-platform when not needed. Alternatively, such drop leaves, alternatively termed platform wings, may be attached or detached as needed.

### OTHER EMBODIMENTS

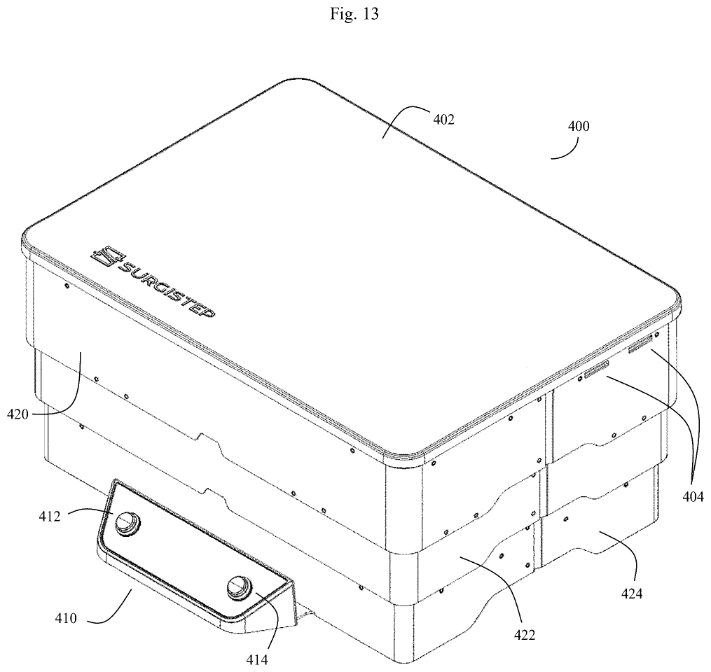

In some embodiments, as shown in FIG. 13-15, the motorized step-platform device may further comprise a user interface (410), a motorized height adjustment device, a height sensor, top plate (402), at least two telescoping skirts (such as 420, 422, 424), and a bottom plate (406). In this embodiment, the device may be mounted on at least three retractable ball caster assemblies (440). Each ball caster assembly may comprise a retractable ball caster (442) mounted in a holder (444), and configured with a spring arrangement (446) that allows the retractable ball caster (442) to extend out from the holder (444) and bottom plate (406) when the weight on the top plate (402) is less than a preset amount (usually when the operator is not standing on the top plate). This allows the step platform device to be moved by an external force.

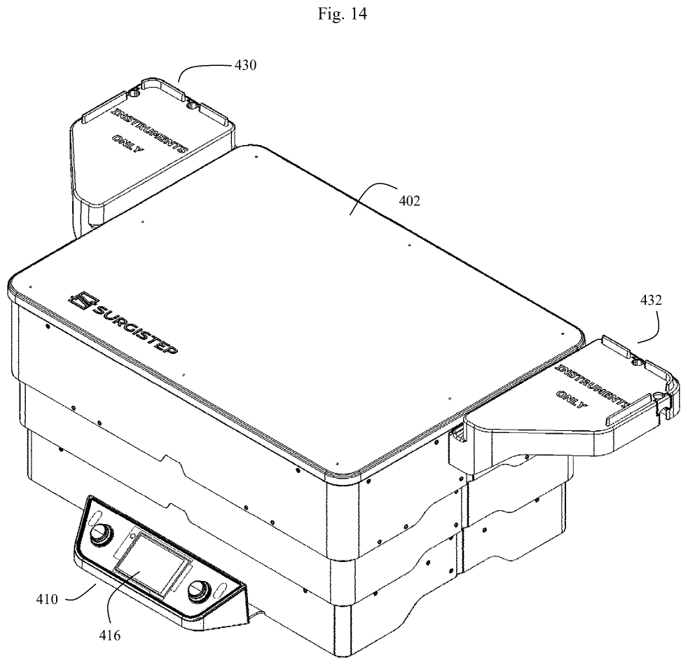

FIG. 13 shows a top view of an alternate embodiment of the surgical step stool (400), here with a simplified top plate (402), and a foot-activated user interface (410) comprising at least two switches (412, 414). Note the optional slots (404) in the upper telescoping skirt (420), which can accommodate tabs (see FIG. 14A, 433) to mount optional side platform wings (430, 432) as shown in FIG. 14.

The middle and lower telescoping skirts are shown as (422) and (424).

FIG. 14 shows a top view of an alternate embodiment of the surgical step stool previously shown in FIG. 13, here further comprising two optional side platform wings (430, 432). These side platform wings can be used to hold foot pedals, such as (210, 212), which may be used to control other operating room equipment. Note that in FIG. 14, the user interface (410) also has an optional processor-driven display (416) (e.g., a display screen). In some embodiments, this may be a touch-sensitive display screen.

However, when the operator stands on the top plate (402), the ball caster assembly is configured with a spring arrangement to detect that the weight exceeds a preset amount. As a result, the ball caster (442) retracts into its holder (444) and, in turn, into the bottom plate (406). This increases friction between the bottom of the device and the floor, thus impeding any sliding caused by external force when the operator is standing on the device.

FIG. 14A shows a detail of the right-side platform wing (432) previously shown in FIG. 14. The tabs (433) previously discussed in FIG. 13 above is also shown.

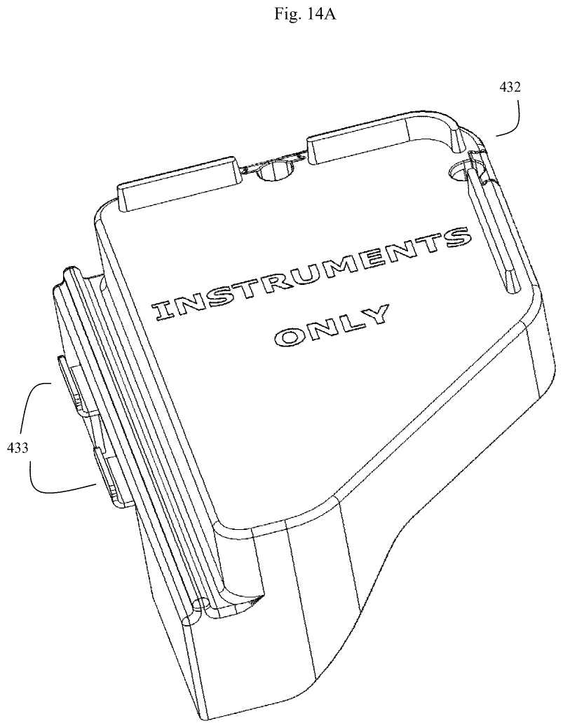

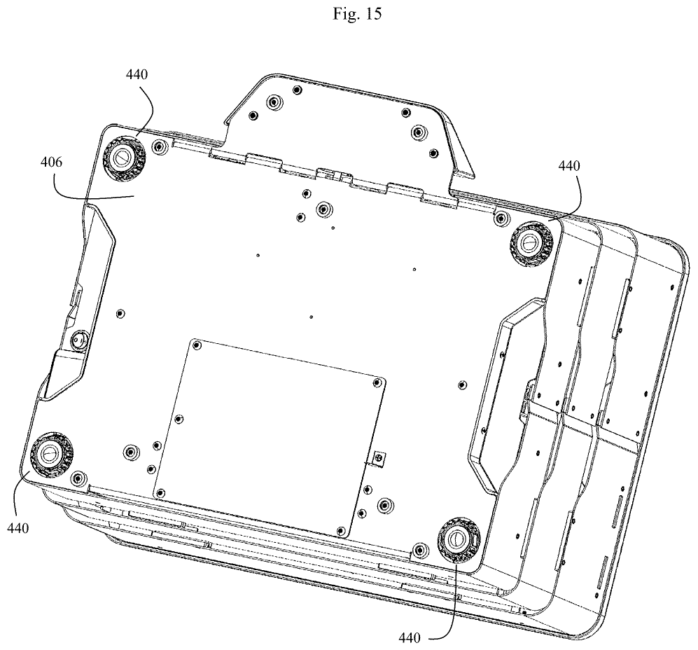

FIG. 15 shows a bottom view of an alternate embodiment of the surgical step stool (400). In this alternate embodiment, the motorized bottom suction device (130, 308, etc.) is optional. Instead, the surgical step stool is supported by at least three retractable ball caster assemblies (440).

FIG. 16 shows a detail of one of the retractable ball caster assemblies (440) previously shown in FIG. 15. In these assemblies (440) each retractable ball caster (442) is mounted in a holder (444), and configured with a spring arrangement (446) with a spring force and range of motion selected to allows the retractable ball caster (442) to extend from the holder (444) and the bottom plate (406) when no operator is on the step stool (e.g. added operator weight less than a preset amount, such as less than 30-50 pounds). Thus, when the only weight on the retractable ball casters is merely the weight of the step stool itself (usually 40-50 pounds), the spring forces the ball caster out. This allows the ball caster (442) to spin freely, enabling the step stool to be moved or slid to another position as desired. The spring arrangement (446) is further configured so that when an operator stands on the step stool (e.g., with an operator weight exceeding a preset amount, such as more than 40-50 pounds), full weight on the ball casters is thus the weight of the device plus the operator's weight, which is usually more than about 100 pounds. Under this amount of pressure, the ball caster spring is configured to enable the ball caster to retract into the holder. This allows the holder (444) or other higher-friction portions of the assembly (448) to come into contact with the floor. This increases friction between the step stool and the floor, impeding further motion of the step stool.

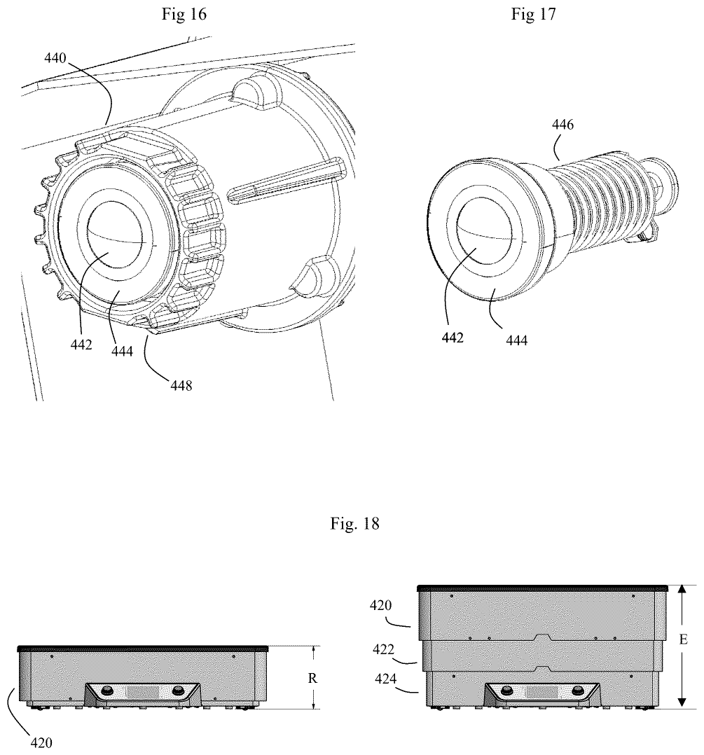

FIG. 17 shows an interior detail of the retractable ball caster assembly (440), showing the spring arrangement (446) in more detail.

In some embodiments, at least one processor (300) is configured to use input from either the memory or from a user interface (410), and optional input from a height sensor, to height-adjust the step-platform device to at least a first height by commanding the motorized height adjustment device to adjust the height of the step-platform device by adjusting an elevation of the top plate (402) above the bottom plate (406).

In some embodiments, at least one of the telescoping skirts (e.g. 420) is attached to the top plate (402), and at least one of the telescoping skirts (e.g. 424) is attached to the bottom plate (406). Here, the overlap between at least some of these telescoping skirts (420, 422, 424) changes in response to the elevation. This is shown in FIG. 18.

FIG. 18 shows examples of the step stool in the retracted (R) and extended (E) configurations. Note that in this example, the step stool has three telescoping skirts (420, 422, 424). Here, in the retracted configuration, the top telescoping skirt (420) at least partially covers the lower telescoping skirts.

In some embodiments, as shown in FIG. 7, FIG. 13, and FIG. 14, the user interface (410) comprises a display (416) and at least one control switch (412), and the at least one processor (300) is configured to transmit and receive any of commands and data from the user interface (410).

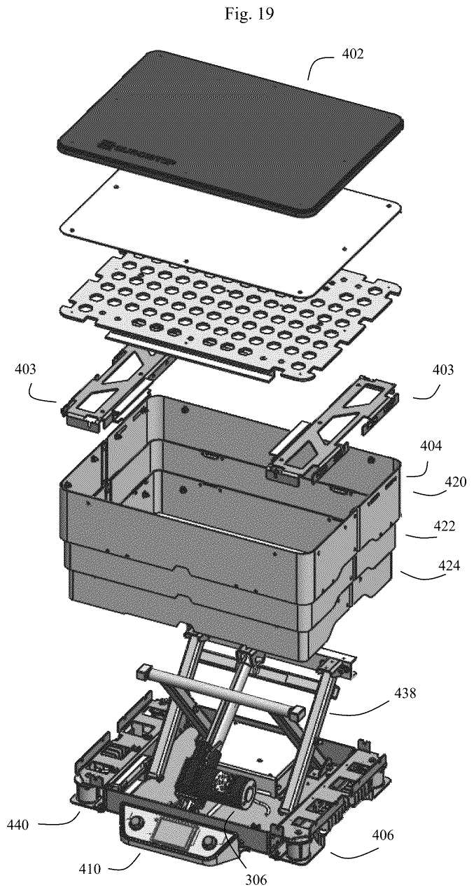

In some embodiments, as shown in FIG. 14 and FIG. 19, any of the top plate (402) and at least one of the telescoping skirts (such as 420), suitable slots (404) in the skirts, as well as internal structural brackets (403) may be configured to reversibly attach to at least one removable side platform wing module (430, 432) configured to extend the surface area of the top plate. Here, in a preferred embodiment, at least one of these side platform wings (430, 432) are further configured for foot pedal (210, 212) containment. In a preferred embodiment, these side platform wing modules are designed for toolless attachment and removal.

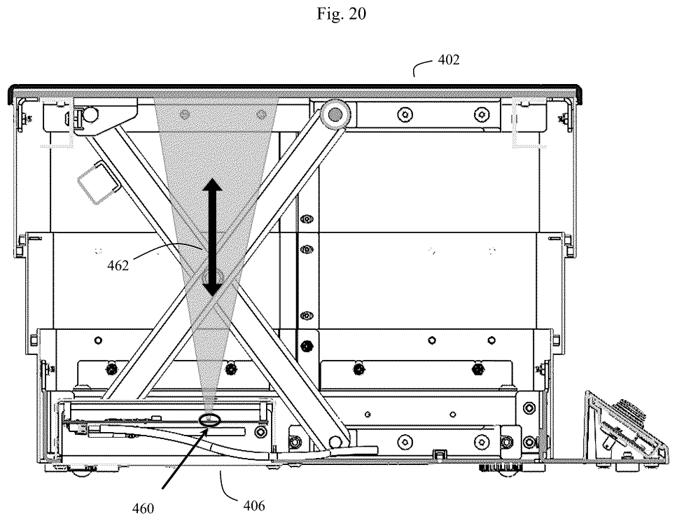

As shown in FIG. 7, FIG. 20, and elsewhere, in some embodiments, the height sensor comprises any of a time-of-flight sensor (460), feedback from an actuator used to control the motorized height adjustment device (306), or other motion or height sensor.

FIG. 19 shows an exploded diagram of the alternate embodiment of the invention previously shown in FIG. 13. Here, structural bracket(s) (403), which may be used to provide structural support to the platform wings (430, 432) as they attach to the device, are shown. The side slots (404) are also shown.

As shown in FIG. 19, in some embodiments, the motorized height adjustment device comprises a scissor frame (438), and the motorized actuator (306) may be a ball screw type actuator.

FIG. 20 illustrates one example of how a height sensor, comprising an optical time-of-flight sensor in this case, may operate. In this example, the time-of-flight sensor (460) is mounted near (e.g., close to or proximate to) the device's bottom plate, shines a light (462) on the bottom side of the top plate (402), and determines height by measuring the time it takes for the reflected light to return to the time-of-flight sensor. Other methods of determining height, such as measuring a change in position or operation of the motorized actuator (306), alternate distance sensor arrangements, etc., may also be used.

In some embodiments, in addition to comprising retractable ball caster assemblies (440), the device may further comprise at least one motorized bottom suction device such as (130, 308) previously described. Here, at least one processor (300) may be further configured to use input from the user interface (410) or other data interface (304) to command at least one motorized bottom suction device to generate a partial vacuum against the floor as desired. Here, the at least one processor (300) may be further configured to use input from the user interface or other data interface to command this at least one motorized bottom suction device to release this partial vacuum against the floor, again as desired.

1. A motorized step-platform device configured to adjust to operator-to-operator variations, comprising:
a lightweight motorized step-platform device configured to slide along a floor by an application of external force to a first desired position proximate a table;
said motorized step-platform device comprising at least one processor, memory, user interface, motorized height adjustment device, height sensor, top plate, at least two telescoping skirts, and bottom plate;
wherein said top plate is configured to bear a human operator's weight;
said device mounted on at least three retractable ball casters, each said retractable ball caster mounted in a holder, and configured with a spring arrangement that allows each retractable ball caster to extend from said holder when said weight is less than a preset amount, thus enabling said sliding by said external force, and to retract into said holder when said weight is greater than a preset amount, thus impeding said sliding by said external force;
said at least one processor configured to use input from either said memory or from said user interface, and input from said height sensor, to height-adjust said step-platform device to at least a first height by commanding said motorized height adjustment device to adjust said height of said step-platform device by adjusting an elevation of said top plate above said bottom plate;
wherein at least one of said telescoping skirts is attached to said top plate, and at least one of said telescoping skirts is attached to said bottom plate, and wherein overlap between at least some of said telescoping skirts changes in response to said elevation.

2. The step-platform device of claim 1, wherein said top plate is a customizable top plate, and said step-platform is configured to enable toolless attachment and detachment of said customizable top plate from said step-platform.

3. The step-platform device of claim 2, wherein said customizable top plate further comprises at least one defined location, each said at least one defined location configured to interface with an electronic foot switch configured to control electronic equipment;
said interface comprising any of a countersunk depth configured to accommodate said electronic foot switch, a wireless charger configured to recharge said electronic foot switch, or a wired jack or plug configured to receive wired electrical signals from said electronic foot switch;
wherein said table is an operating table, and said electronic foot switch is configured to control operating room equipment.

4. The step-platform device of claim 1, wherein said user interface comprises a display and at least one control switch; and
said at least one processor is configured to transmit and receive any of commands and data from said user interface.

5. The step-platform device of claim 4, wherein said at least one processor and said user interface is configured to transmit or receive operator specific parameters from said memory, said operator specific parameters comprising operator preferred step-platform height parameters.

6. The step-platform device of claim 1, wherein said motorized step-platform comprises at least one rechargeable battery and battery access compartment, and said at least one rechargeable battery and battery access port are configured to enable toolless exchange of said at least one rechargeable battery.

7. The step-platform device of claim 1, wherein any of said top plate, structural bracket, and at least one of said telescoping skirts is further configured to attach to at least one removable side platform wing module configured to extend a surface area of said top plate.

8. The step-platform device of claim 7, wherein at least one of said side platform wings are further configured for foot pedal containment.

9. The step-platform device of claim 1, wherein said height sensor comprises any of a time-of-flight sensor, feedback from an actuator used to control said motorized height adjustment device, or other motion or height sensor.

10. The step-platform device of claim 1, wherein said motorized height adjustment device comprises a scissor frame and ball screw type actuator.

11. The step-platform device of claim 1, wherein said device further comprises at least one motorized bottom suction device;
wherein said at least one processor is further configured to use input from said user interface to command said at least one motorized bottom suction device to generate a partial vacuum against said floor; and
wherein said at least one processor is further configured to use input from said user interface to command said at least one motorized bottom suction device to release said partial vacuum against said floor.

12. A motorized step-platform device configured to adjust to operator-to-operator variations, comprising:
a lightweight motorized step-platform device configured to slide along a floor by an application of external force to a first desired position proximate a table;
said motorized step-platform device comprising at least one processor, memory, user interface, motorized height adjustment device, height sensor, top plate, at least two telescoping skirts, and bottom plate, and at least one motorized bottom suction device;
wherein said top plate is configured to bear a human operator's weight;
said device mounted on at least three retractable ball casters, each said retractable ball caster mounted in a holder, and configured with a spring arrangement that allows each retractable ball caster to extend from said holder when said weight is less than a preset amount, thus enabling said sliding by said external force, and to retract into said holder when said weight is greater than a preset amount, thus impeding said sliding by said external force;
said at least one processor configured to use input from either said memory or from said user interface, and input from said height sensor, to immobilize said at least one motorized bottom suction device to generate a partial vacuum against said floor, and height-adjust said step-platform device to at least a first height by commanding said motorized height adjustment device to adjust said height of said step-platform device by adjusting an elevation of said top plate above said bottom plate;
wherein said at least one processor is further configured to use input from said user interface to command said at least one motorized bottom suction device to release said partial vacuum against said floor; and
wherein at least one of said telescoping skirts is attached to said top plate, and at least one of said telescoping skirts is attached to said bottom plate, and wherein overlap between at least some of said telescoping skirts changes in response to said elevation.

13. The step-platform device of claim 12, wherein said user interface comprises a display and at least one control switch; and
said at least one processor is configured to transmit and receive any of commands and data from said user interface.

14. The step-platform device of claim 13, wherein said at least one processor and said user interface is configured to transmit or receive operator specific parameters from said memory, said operator specific parameters comprising operator preferred step-platform height parameters.

15. The step-platform device of claim 12, wherein said motorized step-platform comprises at least one rechargeable battery and battery access compartment, and said at least one rechargeable battery and battery access port are configured to enable toolless exchange of said at least one rechargeable battery.

16. The step-platform device of claim 12, wherein any of said top plate, structural bracket, and at least one of said telescoping skirts is further configured to attach to at least one removable side platform wing module configured to extend a surface area of said top plate.

17. The step-platform device of claim 16, wherein at least one of said side platform wings are further configured for foot pedal containment.

18. The step-platform device of claim 12, wherein said height sensor comprises any of a time-of-flight sensor, feedback from an actuator used to control said motorized height adjustment device, or other motion or height sensor.

19. The step-platform device of claim 12, wherein said motorized height adjustment device comprises a scissor frame and ball screw type actuator.
